(19) United States
(12) Patent Application Publication
(10) Pub. No.: US 2011/0306045 A1
(43) Pub. Date: Dec. 15, 2011

(54) Method for Confirming a Diagnosis of Rolandic Epilepsy

(75) Inventors: Deb Kumar Pal, London (GB); Lisa Joanna Strug, Toronto (CA)

(73) Assignees: THE TRUSTEES OF COLUMBIA UNIVERSITY IN THE CITY OF NEW YORK, NEW YORK, NY (US); THE HOSPITAL FOR SICK CHILDREN, Toronto, ON (CA)

(21) Appl. No.: 13/141,044

(22) PCT Filed: Dec. 21, 2009
(86) PCT No.: PCT/US09/68988

§ 371 Date: Aug. 15, 2011

(30) Foreign Application Priority Data
Dec. 19, 2008 (US) 61/139486

(51) Int. Cl. C12Q 1/68

(52) U.S. Cl. 435/6.11

(57) ABSTRACT

A strong association between variants in Elongator Protein Complex 4, (ELP4) (specifically single nucleotide polymorphisms, SNPs) at the 11p13 locus on chromosome 11 and the centrotemporal sharp wave trait (CTS) has been discovered, which association has diagnostic significance for rolandic epilepsy. It has further been discovered that the 11p13 locus has a pleiotropic role in the development of speech motor praxis and CTS, which supports a neurodevelopmental origin for classic rolandic epilepsy (RE).

### CROSS REFERENCE TO RELATED APPLICATIONS

This application claims benefit of Provisional Application 61/139,486, filed on Dec. 19, 2008, incorporated herein by reference, under 35 U.S.C. §119(e).

### STATEMENT OF GOVERNMENTAL INTEREST

This invention was made with Government support under National Institutes of Health grants NS047530 (DKP), HG00-4314 (LJS) and NS27941 (DAG). The Government has certain rights in the invention.

### BACKGROUND OF THE INVENTION

1. Field of the Invention

This invention is in the field of diagnosing rolandic epilepsy.

2. Description of the Related Art

Rolandic epilepsy (RE) is the most common human epilepsy, affecting children between 3 and 12 years of age, boys more often than girls (3:2). Focal sharp waves in the centrotemporal area define the electroencephalographic (EEG) trait for the syndrome. Focal sharp waves are a feature of several related childhood epilepsies and are frequently observed in common developmental disorders including speech dyspraxia, attention deficit hyperactivity disorder (ADHD) and developmental coordination disorder (DCD). Epilepsy is a very common brain disorder characterized by recurrent seizures, resulting from abnormal nerve cell activity in the brain. Some cases of epilepsy are caused by brain pathology, such as stroke, infection, tumor, or head injury. Others—so called “idiopathic”—do not have a clear cause and are presumed to have a genetic basis. Rolandic epilepsy is the most common idiopathic human epilepsy and affects children, mostly boys. It has an electroencephalographic signature that is also found in multiple neuro-developmental disorders, many of which may be co-morbidities of RE.

Rolandic epilepsy (MIM 117100) is a neuro-developmental disorder, affecting 0.2% of the population, characterized by classic focal seizures that recapitulate the functional anatomy of the vocal tract, beginning with guttural sounds at the larynx, sensorimotor symptoms then progressing up to the tongue, mouth and face, culminating with speech arrest. Seizures most often occur in sleep shortly before awakening. The disorder occurs more often in boys than girls (3:2) and is diagnosed in 1 in 5 of all children with newly diagnosed epilepsy [1]. All patients exhibit the defining EEG abnormality of centrotemporal sharp waves (CTS). The onset of seizures in childhood (3-12 years) [2] is frequently preceded by a constellation of developmental deficits including speech disorder, reading disability and attention impairment. These deficits have been noted to cluster in family members of RE patients who do not have epilepsy [3, 4]. None of these abnormalities are associated with major cerebral malformations visible on routine MRI [5]. The seizures and the EEG abnormality of centrotemporal sharp waves spontaneously remit at adolescence, although the prognosis for developmental deficits is less clear. There is no known involvement of organs outside the nervous system.

The focal sharp waves of RE include some that are characterized by more severe and varied types of seizures (Atypical Benign Partial Epilepsy or ABPE, MIM 604827); variable locations (Benign Occipital Epilepsy, MIM 132090); acquired receptive aphasia (Landau-Kleffner syndrome, MIM 245570); and developmental regression (Continuous Spikes in Slow-Wave Sleep). CTS are common in children (2-4%) [6], have equal gender distribution, and have been observed with increased frequency in developmental disorders, including speech dyspraxia [7], attention deficit hyperactivity disorder (ADHD) [8], and developmental coordination disorder (DCD) [9], showing that the EEG trait of CTS is not specific to epilepsy, but possibly a marker for an underlying subtle but more widespread abnormality of neurodevelopment [10].

Despite the strong clustering of developmental disorders in RE families, RE itself has a low sibling risk of ˜10% [11]. Several rare, phenotypically distinct Mendelian RE variants have been reported [12-15], but the common form appears to have complex genetic inheritance. However, segregation analysis shows that CTS in the common form of RE is inherited as an autosomal dominant trait [16]. CTS were reported to link to 15q14 in a candidate gene study of families multiplex for RE and ABPE, but this locus has not been replicated, and no genome wide screen for CTS has been previously attempted [17]. There is still a need for a diagnostic assay for RE, also understanding the mechanism of CTS could provide insight into the variety of common neuro-developmental disorders in which CTS are observed. We therefore set out to genetically map the CTS trait in RE families.

### BRIEF DESCRIPTION OF THE DRAWINGS

The present invention is illustrated by way of example, and not by way of limitation, in the FIG.s of the accompanying drawings, in which:

FIG. 1. (FIG. A) Multipoint LOD and heterogeneity LOD (HLOD) scores for CTS on chromosome 11: maximum HLOD=4.3 at D11S914 under a dominant mode of inheritance with 50% penetrance; (FIG. B) SSD/EEG dominant model with 50% penetrance, max at D11S914 (48 cM).

FIG. 2. Cochrane-Armitage Trend test of case-control association for CTS at the 11p13 locus in the discovery (New York) and replication (Calgary) data sets: Bonferroni critical value lines displayed for the two datasets; significance criteria of 0.05/30 in replication set and 0.05/44 in discovery set corresponding to the 30 and 44 SNPs evaluated in the two analyses.

FIG. 3. For linkage analysis, affectedness data and DNA were collected from all potentially informative and consenting relatives of the proband. In most cases this included at least both parents and all siblings over the age of 3 years. Pure likelihood plot of association evidence in discovery set (FIG. 3A) and in joint analysis of datasets (FIG. 3b). This pure likelihood analysis plots odds ratio (OR) on the y-axis and base pair position on the x-axis. Each vertical line represents a Likelihood Interval (LI) for the OR at a given SNP. The OR=1 line is plotted as a solid black horizontal line, for reference. LIs in color are denoted as SNPs of interest, while a grey line indicates that the SNP is not of interest because the 1/32 LI for that SNP covers the OR=1 line. The small horizontal tick on each LI is the maximum likelihood estimator for the OR. The portion of the colored LI that covers the OR=1 horizontal line indicates the strength of the association information at that SNP. In particular, if the green portion is above the OR=1 line while the red portion of the LI covers the OR=1 line, then the LOD evidence at that SNP is between 1.5 and 2 (i.e. the 1/32 LI does not include the OR=1 value, but the 1/100 LI does); similarly, if both the red and green portions are above the OR=1 line but the blue portion covers the line, then the LOD evidence is between 2 and 3 (i.e. the 1/100 LI does not include OR=1 as a plausible value but the 1/1000 LI does). The further the colored line is above the OR=1 line, the stronger the association evidence. The max LR for each SNP in color is also provided as text in the plot, providing evidence not only of whether the LOD-evidence is between 2 and 3, but also the exact value of the max LR.

### DETAILED DESCRIPTION

It has been discovered through a genomewide linkage analysis that the centrotemporal sharp waves (CTS) trait in 38 US families singly ascertained through a RE proband using a pure likelihood statistical analysis that CTS maps to variants in Elongator Protein Complex 4, ELP4 on chromosome 11p13 (Multipoint LOD 4.30). Elongator depletion results in the brain-specific downregulation of genes implicated in cell motility and migration. DNA collection, STR genotyping, linkeage analysis, SNP markers, SNP genotyping and resequencing methods are described in Example 1. (62). Further studies indicated that CTS in RE patients and families were associated with SNP markers rs986527 and rs964112, and rs1232182 in the 11p13 linkage region in two independent datasets. Resequencing of ELP4 coding, flanking and promoter regions revealed no significant exonic polymorphisms. The strong association between variants in Elongator Protein Complex 4, (ELP4) (specifically single nucleotide polymorphisms, SNPs) at the 11p13 locus on chromosome 11 and the centrotemporal sharp wave trait (CTS), have diagnostic significance for rolandic epilepsy. Based on these results new methods of confirming a diagnosis of RE and determining if a patient has an increased risk of developing RE have been discovered.

Applicants have further discovered that the 11p13 locus has a pleiotropic role in the development of speech motor praxis and CTS, which supports a neurodevelopmental origin for classic rolandic epilepsy (RE). Data from experiments using computerized acoustic analysis of recorded speech showed abnormalities in voice-onset time and vowel duration in RE probands, siblings and parents, providing evidence of breakdown in the spatial/temporal properties of speech articulation consistent with a dyspraxic mechanism is also linked to the 11p13 locus (based on a CTS/SSD (Max multipoint LOD 7.50 at D11S914) phenotype).

Without being bound by theory, we hypothesize that an as yet unidentified, non-coding mutation in linkage disequilibrium with SNPs in ELP4 impairs brain-specific Elongator mediated interaction of genes implicated in brain development, resulting in susceptibility to seizures, speech dyspraxia and neurodevelopmental disorders.

An embodiment of the invention is directed to a method for confirming a diagnosis of rolandic epilepsy in a patient who has had a seizure or an interictal EEG with centrotemporal sharp waves and normal background includes:
a. obtaining a DNA sample from the patient,
b. analyzing the DNA sample to determine if there is a nucleotide variant in the Elongator Protein Complex 4 (ELP4) gene on chromosome 11, and
c. if the nucleotide variant is detected, concluding that the patient has rolandic epilepsy.

In an embodiment the gene locus is the 11p13 locus, and the variant is an SNP that is a member of the group comprising rs964112s in intron 9, rs1232182 in intron 5 and rs986527 in intron 5 of the Elongator Protein Complex 4 gene. In other embodiments the method further includes one or more of the following steps: determining if the patient has a parent or sibling with rolandic epilepsy, determining if the patient has a developmental deficit that is a member selected from the group comprising a speech sound disorder such as speech dyspraxia, a reading disability, a developmental coordination disorder (DCD) and an attention impairment such as attention deficit hyperactivity disorder (ADHD), which further aid in confirming the diagnosis. The patient's DNA sample is derived from any patient cell type, preferably cells selected from the group comprising white blood cells, saliva leukocytes, lymphoblasts, epidermal cells, and fibroblasts.

The method optionally includes confirming that the neuroimaging of the patient's brain excludes an alternative structural, inflammatory or metabolic cause for the seizure or the interictal EEG with centrotemporal sharp waves and normal background. The confidence of the diagnosis of RE is strengthened if it is determined that the patient is under 15 years of age.

Similar methods can be used to determine if a patient has an increased risk of developing rolandic epilepsy. Specifically, the patient has an increased risk of developing RE if the DNA sample shows nucleotide variant rs964112s in intron 9, rs1232182 in intron 5 or rs986527 in intron 5 of the Elongator Protein Complex 4 gene. By an increased risk is meant that the patient has a risk of developing RE that is statistically significant compared to the general population that does not have the mutation.

Example 1 describes the details of experiments showing that mutations in the ELP4 subunit of Elongator are associated with the pathogenesis of rolandic epilepsy, and have a strong effect on risk for CTS in RE families. This locus appears to be distinct from those discovered in rare Mendelian RE variants. The precise mutation that is in linkage disequilibrium with the associated SNPs in ELP4 remains to be determined; however, the data show that it lies either in the non-coding regions of ELP4, or possibly just beyond the gene. This finding represents the first susceptibility gene identified for a common idiopathic focal epilepsy, and the first step in unlocking the complex genetics of RE and related childhood epilepsies. It is also the first reported disease association with ELP4 in humans, and offers possible insights into the etiology and kinship of associated developmental cognitive and behavioral disorders.

ELP4 is one of six subunits (ELP1-ELP6) of Elongator [37], which has both nuclear and cytoplasmic localization and two distinct but incompletely characterized roles in eukaryotic cells [38]: in transcription [39] and in tRNA modification [40]. Elongator associates with and regulates RNA Polymerase II (RNAPII), and is important for assisting the transcription complex along the template during transcript elongation, arguably by catalyzing histone H3 acetylation [41]. Elongator plays a key role in transcription of several genes that regulate the actin cytoskeleton, cell motility and migration [42]. These functions are crucial in the nervous system for nerve cell growth cone motility, axon outgrowth and guidance, neuritogenesis and neuronal migration during development. Depletion of Elongator results in cell migration defects in neuronal cells, although it is not clear if these are mediated via transcript elongation of target genes (e.g. beclin-1, gelsolin) [42], or by direct cytosolic association with filamin A and dynein heavy chain proteins in membrane ruffles [43]. Other Elongator subunit mutations have been implicated in human neurological disease such as Riley-Day syndrome (MIM 223900) that is an autosomal recessive, sensory and autonomic neuropathy, with EEG abnormalities and epilepsy [44-47].

Until now mechanism for RE associated speech sound disorder was not known. The results of experiments set forth in Example 2 show that speech dyspraxia is the neural mechanism for the speech sound disorder that is comorbid in RE families. Dyspraxia refers to a motor planning impairment that is not caused by weakness, ataxia, sensory loss or difficulty in task comprehension. In the verbal sphere, developmental verbal dyspraxia refers to a “phonological disorder resulting from a breakdown in the ability to control the appropriate spatial/temporal properties of speech articulation” (38, 39). The genetic analyses further show that there are pleiotropic effects of the 11p13 locus for both speech sound disorder and the abnormal CTS electroencephalographic pattern seen in Rolandic epilepsy patients and their families. Together, these findings show a basis for the pathogenesis for seizure susceptibility, and implicate a developmental basis for seizure susceptibility and comorbidity in RE.

Using the highly sensitive method of acoustic analysis to detect subclinical impairments in speech motor coordination, it was discovered that a mild form of developmental verbal dyspraxia explains the speech sound disorder that is commonly found in classic RE cases and that aggregates among their relatives (7). Voice-onset time and vowel duration abnormalities were detected in 13/18 RE probands, 14/16 siblings and 8/15 parents, providing evidence of a breakdown in the spatial/temporal properties of speech articulation that is consistent with a dyspraxic mechanism. In two-point lodscore analysis, evidence for linkage to the 11p13 locus was found when the phenotype qualification for the study was broadened from CTS to CTS/SSD (Max LOD 4.30 at D11S4102). In multipoint lodscore analysis, maximum linkage evidence of 7.50 was obtained for CTS/SSD at D11S914.

Certain embodiments of the invention include determining if a subject has a speech dyspraxia to confirm a diagnosis or increased risk for developing RE. Methods such as those described in Example 2 can be used to test for speech dyspraxia.

### EXAMPLES

### Example 1

### CTS Mapped to Variants in Elongator Protein Complex 4, ELP4 on Chromosome 11

A genomewide linkage analysis of the centrotemporal sharp waves (CTS) trait in 38 US families singly ascertained through a RE proband was conducted using a pure likelihood statistical analysis to map CTS to variants in Elongator Protein Complex 4, ELP4 on chromosome 11. In 11 of the 38 families, one additional sibling was known to carry the CTS trait, but the CTS status of individuals younger than 4 years or older than 16 years was unknown because of its age-limited expression. The maximum two-point and multipoint LOD scores for CTS were observed at 11p13. The 13cM linkage region encompassing the area in which LOD scores >2.0 was designated as the region of interest for fine mapping. Association of CTS with SNP markers distributed across genes in this region was determined initially using a “discovery” dataset that included 68 cases and 187 controls group-matched for ancestry and gender—38 of these cases were included in the original linkage screen. In addition to case-control analysis, family-based analysis was used to guard against the potential for positive confounding due to population stratification. A pure likelihood approach to the statistical analysis of linkage and association [18-21], was used. Additional SNPs around genes that showed compelling evidence of association in the preliminary analysis were then typed.

Subjects. Informed consent was obtained from all participants using procedures approved by institutional review boards at each of the clinical research centers collecting human subjects. The general methodology for the study has been detailed elsewhere [3]. Briefly, cases with classic rolandic epilepsy and their families were recruited for a genetic study from eight pediatric neurology centers in the northeastern USA (see Acknowledgements for referring physicians). Ascertainment was through the proband, with no other family member required to be affected with RE. All cases were centrally evaluated by a pediatric neurologist, as well as by one other study physician. After evaluation, cases were enrolled if they met stringent eligibility criteria for RE, in accordance with the definition of the International League Against Epilepsy [22] including:
(i) at least one witnessed seizure with typical features: nocturnal, simple partial seizures affecting one side of the body, or on alternate sides;
(ii) oro-facial-pharyngeal sensorimotor symptoms, with speech arrest and hypersalivation;
(iii) age of onset between 3-12 years;
(iv) no previous epilepsy type;
(v) normal global developmental milestones;
(vi) normal neurological examination;
(vii) at least one interictal EEG with centrotemporal sharp waves and normal background, verified by two independent and blinded readers [16]; and
(viii) neuroimaging read by two independent and blinded board-certified neuroradiologists that excluded an alternative structural, inflammatory or metabolic cause for the seizures [5].

Thus, cases with unwitnessed episodes or with only secondary generalized seizures were excluded, even if the EEG was typical. Siblings between the ages of 4 and 15 years underwent sleep-deprived EEGs to assess their CTS status [16]; EEGs were then evaluated blind to identity by two independent experts

“Cases had their first seizure at a median age of 8 years (range 3-12); most had less than 10 lifetime seizures; over a third had at least one secondary generalized seizure, but only two had a history of convulsive status epilepticus; and two-thirds had been treated with antiepileptic drugs. Table 1 shows the seizure characteristics of the cases. Cases were 60% male and 76% European ancestry (Table 2). Details of EEG and imaging findings have previously been reported [5, 16].

TABLE 1
CLINICAL DESCRIPTORS OF RE CASES

| Feature, % | Discovery Set | Replication Set |
|---|---|---|
| Febrile seizures | 4 | 17 |
| Right handedness | 86 | 93 |

Usual laterality of seizure

| | | |
|---|---|---|
| Left | 36 | 22 |
| Right | 32 | 17 |
| Inconsistent | 32 | 61 |

Predominant EEG lateralization

| | | |
|---|---|---|
| Left | 29 | 20 |
| Right | 53 | 41 |
| Bilateral | 11 | 32 |

Lifetime seizure total

| | | |
|---|---|---|
| ≦10 | 70 | 59 |
| >10 | 30 | 41 |

Relation of seizures to sleep or drowsiness

| | | |
|---|---|---|
| Exclusive | 89 | 83 |
| Not exclusive | 11 | 17 |
| Ever treated with antiepileptic drugs | 70 | 66 |
| Developmental speech delay | 38 | 20 |
| Reading disability | 52 | 34 |
| Migraine headaches | 20 | 20 |

TABLE 2
SELF-REPORTED ANCESTRAL BACKGROUNDS OF PROBAND
CASES AND CONTROLS

| | Discovery set | | | | Replication set | | | |
|---|---|---|---|---|---|---|---|---|
| | (New York) | | | | (Calgary) | | | |
| Ancestry of Proband | Cases | | Controls | | Cases | | Controls | |
| European | 52 | (76) | 132 | (71) | 34 | (85) | 103 | (86) |
| Asian | 2 | (3) | 21 | (11) | 2 | (5) | 10 | (8) |
| African/African- | 3 | (4) | 5 | (3) | 0 | | 0 | |
| American | | | | | | | | |
| Middle Eastern | 0 | | 4 | (2) | 1 | (2) | 2 | (2) |
| Mixed | 8 | (12) | 13 | (7) | 4 | (10) | 3 | (3) |
| Caribbean-Latin | 3 | (4) | 10 | (5) | 0 | | 1 | (1) |
| French-Canadian | 0 | | 0 | | 0 | | 1 | (1) |
| Total | 68 | | 187 | | 40 | | 120 | |

For linkage analysis, affectedness data and DNA were collected from all potentially informative and consenting relatives of the proband. In most cases this included at least both parents and all siblings over the age of 3 years.

Controls. 187 controls were recruited from the same geographic locations as the cases and were group matched for gender and ancestry (see Table 2). Each potential control was screened for personal and family history of neuropsychiatric and developmental disorders: DNA from individuals with a history of seizures was excluded from the control panel. The lifetime CTS status of controls was unknown because of their developmental expression, but assumed to be representative of the general population [6] i.e. 2-4%; thus any observed association in case-control analysis should be conservative. The sample of independent cases and controls is referred to throughout as the discovery dataset.

Replication set. 40 cases and 120 controls were recruited from Calgary, Canada according to the same eligibility criteria as in the discovery dataset (Table 2). The cases were 56% male and 83% of European ancestry, with median age of seizure onset at 7 years. Controls were also 56% male and 86% of European ancestry. Information regarding personal and family history of neuropsychiatric and developmental disorders was collected as above for possible exclusion from case-control analysis. The Calgary sample is referred to as the “replication dataset”.

Association Analysis. Pure Likelihood vs. Frequentist Analysis

We conducted a pure likelihood analysis of the SNP data [18, 19] as well as calculating standard frequentist Bonferroni adjusted p-values for comparison. The two methods provide the same ordering of importance for SNPs. However, they have different significance thresholds, different sample size requirements and different approaches to the adjustment for multiple hypothesis testing. A pure likelihood display of the data provides a more visually informative understanding than standard plots of kb by −log 10 (p-value). Moreover a pure likelihood analysis is particularly well suited for joint analysis of multi-stage designs [27], largely due to how pure likelihood analyses adjust for Type I error inflation due to multiple hypothesis testing.

Pure likelihood analysis provides an objective measure of what a given body of data says about association without the need to incorporate prior information (as required by Bayesian analysis), or interpret association evidence within the context of what would have been seen over multiple replications of the same experiment (Frequentist analysis). The pure likelihood approach also provides a way to control the probability of observing weak signals in the data, and provides an intuitive approach to multiple test adjustments. Therefore, the pure likelihood analysis was used to determine our SNPs of interest for follow-up. We provide p-values for those unfamiliar with pure likelihood analysis, for comparison only. Adjustments for multiple SNP tests are accomplished by following up signals from the first stage with additional samples analyzed in a joint analysis [27]. This is in contrast to standard p-value analysis approaches that require evidence adjustment of p-values, e.g. Bonferroni, FDR [28]. Multiple SNP tests increase the Type I error rate associated with the study (the family-wise error rate), and without p-value adjustments, the type 1 error rate will exceed the fixed rate, e.g. α=0.05.

### Frequentist Methods Use

In the standard frequentist analyses a Cochran-Armitage test for trend in the case-control sample was calculated, requiring Bonferroni corrected critical values for significance. We used a transmission disequilibrium test as implemented in FBAT [29] in the subset of trios to ensure that any signal found through case-control analysis was not itself due to population stratification. For multi-locus analysis, multiple logistic regression of main effects and two-way interactions was used, coding the SNP genotypes as −1, 0 and 1, with their interaction the product of these genotypes at two loci. Haplotypes were constructed using Phase 2.1.1 [30]. To estimate the haplotypes 5000 iterations were used, with 1 thinning interval and 5000 burn-ins. The positions of the markers were not specified. Multiple runs varying the seed were used to determine whether the phase assignments were consistent. Differences in haplotype and haplo-genotype frequencies between cases and controls were determined using chi-square statistics. Odds ratios with 95% confidence intervals were computed.

### Pure Likelihood Methods Used

In a pure likelihood analysis, observed likelihood ratios are reported and figures of likelihood intervals (LI) for the odds ratio, by base pair position are provided. For example, a 1/32 LI is defined as the set of OR values where the standardized likelihood function (divided by the likelihood evaluated at the maximum likelihood estimator) is greater than 1/32 [18]. Likelihood intervals are analogous to confidence intervals in that they are comprised of all parameter values that are supported by the data. However, LIs do not require a long-run frequency interpretation, rather they reflect the evidence about the OR provided by the given dataset. The pure likelihood analysis is presented for the case-control samples only, where a trend disease model is also assumed. For the likelihood analysis, profile likelihoods were used [31] to construct the likelihood ratios in order to eliminate nuisance parameters (i.e. confounding variables) to assess the association evidence at each SNP. We used LOD-evidence of strength 1.5 as a criterion from the observed likelihood ratios to define a SNP of interest.

Type I error. In the pure likelihood paradigm, one does not use error rates such as Type I and II error probabilities for design; instead the probabilities of misleading and weak evidence are controlled at the design phase of the study. For more on the pure likelihood paradigm see [18-21]. Briefly, misleading evidence under the null hypothesis Mₒ is the analogous error rate to a Type I error rate, and measures the rate at which the LR will provide strong evidence favoring the incorrect hypothesis of association, in order to ensure that the probability of observing LOD-evidence of 1.5 favoring association at a SNP of interest, when that SNP is not associated, is very small. Mₒ is generally much smaller than a Type I error [21,32] and over multiple SNP tests, (N=44 for the discovery data set), the family-wise error rate (FWER) is bounded in this particular study by N*Mₒ=0.088. By using our two-stage design this error probability is bounded by 0.044, and consequently the replication phase provides our adjustment for conducting multiple SNP tests. This is because the replication phase ensures that the FWER is controlled at acceptable levels, the whole point of multiple test adjustments. In a Frequentist analysis, if the significance criterion is set at 5%, then the FWER rate is controlled at 0.05. The probability of weak evidence (W)—the probability of obtaining a weak association signal, perhaps between 0.5 and 1.5, when in fact there is association—has no frequentist analog, and should be controlled during the planning phase of a study by choosing sufficient sample size to ensure this error rate remains low. For this study W was quite high, W=0.11 for a given SNP test, and due to the small sample size. However, fortunately, some strong evidence in ELP4 was observed, and the a priori weak evidence probability associated with the study does not detract from the strong conclusions that can be made about the ELP4 CTS association.

DNA collection. DNA was collected either by peripheral venous blood draw into 10 ml K-EDTA tubes (Fisher Scientific), or by salivary sample in ORAGENE (DNA Genotek, Ottawa) flasks. DNA was purified from saliva samples and total white blood cells stored lysed in the Puregene Cell Lysis solution using the Puregene DNA Purification kit. Extracted DNA was dissolved in water. DNA yield was determined by UV spectrophotometry using the Spectramax Plus 96-well microplate reader from Molecular Devices Corp. Absorbance was measured at 260, 280 and 320 nm. DNA concentration was determined from the 260 nm reading and the quality of the extracted DNA was assessed by 260/280 ratio. DNA stock solutions were stored at −80° C.

STR genotyping. Short tandem repeat (STR) loci are polymorphic regions found in the genome that are used as genetic markers for human identity testing. Typing of STR loci by polymerase chain reaction (PCR) is becoming a standard for nuclear DNA genotyping analysis. The 194 individuals from 38 RE families were genotyped using the deCODE 4cM STR marker panel. This panel contains approximately 1200 highly polymorphic STR markers. Amplified fragments were electrophoresed using ABI 3700 and ABI 3730 DNA analyzers with CEPH family DNA used as control. Alleles were called automatically and checked for consistency with Hardy-Weinberg equilibrium and non-paternity. Errors were reconciled by resampling or exclusion of inconsistent genotypes.

Linkage analyses. We analyzed the linkage data by two-point and multipoint heterogeneity LOD score calculations in all 38 families combined. We used the MMLS approach to parametric linkage analysis [23]. Briefly, LOD scores were calculated under both dominant and recessive modes of inheritance, specifying a dominant gene frequency of 0.01 and a recessive gene frequency of 0.14, a sporadic rate of 0.0002, and penetrance of 0.50. In regions providing evidence for linkage, we then maximized over a grid of penetrance values from 0 to 1.0 by 0.05 increments. Marker allele frequencies were calculated from the dataset. In two-point analysis markers were noted that yielded LOD scores greater than 2.0. Two-point results with multipoint analysis using Genehunter [24] were followed up, again using the MMLS approach but maximizing over penetrance and computing heterogeneity LOD scores. A sex-averaged map was used because the observed multipoint LOD scores should be conservative in the presence of linkage, if indeed there are male-female map differences [25]. Simulation results confirmed that differential male-female map distance has little effect on localization of the maximum LOD score (data not shown). Separate analyses were conducted in the European and non-European ancestral subgroups.

TABLE 3
SNPS GENOTYPED IN THIS STUDY

| | | | Physical | | | |
|---|---|---|---|---|---|---|
| | | | map | bp to | | |
| Marker | dbSNP | Alleles | location | next | MAF in | |
| number | number | Minor/Major | (bp) | marker | Controls | Gene/Type |
| 1 | rs1015541 | A/C | 30811481 | 0 | 0.323077 | DCDC5 intron |
| | | | | | | 31 |
| 2 | rs1448938 | T/C | 30849400 | 37919 | 0.444882 | DCDC5 intron |
| | | | | | | 28 |
| 3 | rs273573 | A/C | 30867567 | 18167 | 0.326923 | DCDC5 intron |
| | | | | | | 26 |
| 4 | rs395032 | A/G | 30883776 | 16209 | 0.324427 | DCDC5 intron |
| | | | | | | 20 |
| 5 | rs163881 | T/G | 30904820 | 21044 | 0.326923 | DCDC5 intron |
| | | | | | | 12 |
| 6 | rs7117074 | A/C | 30942663 | 37843 | 0.432 | DCDC5 intron |
| | | | | | | 10 |
| 7 | rs290102 | C/T | 30972073 | 29410 | 0.453125 | DCDC5 intron |
| | | | | | | 10 |
| 8 | rs288458 | G/C | 31007585 | 35512 | 0.096 | DCDC5 intron |
| | | | | | | 10 |
| 9 | rs560395 | G/A | 31044369 | 36784 | 0.454545 | DCDC5 intron 8 |
| 10 | rs621549 | A/C | 31070773 | 26404 | 0.461538 | DCDC5 intron 6 |
| 11 | rs208068 | G/A | 31108520 | 37747 | 0.392308 | * |
| 12 | rs400964 | A/T | 31133836 | 25316 | 0.386719 | * |
| 13 | rs16921914 | A/G | 31167347 | 33511 | 0.305344 | * |
| 14 | rs286651 | T/C | 31186380 | 19033 | 0.39313 | * |
| 15 | rs7937421 | C/T | 31252249 | 65869 | 0.205426 | DCDC1 intron 7 |
| 16 | rs2774403 | A/T | 31277106 | 24857 | 0.392308 | DCDC1 intron 6 |
| 17 | rs12577026 | A/G | 31304419 | 27313 | 0.169231 | DCDC1 intron 3 |
| 18 | rs1547131 | C/T | 31343175 | 38756 | 0.257692 | DCDC1 intron 1 |
| 19 | rs483534 | G/C | 31354718 | 11543 | 0.350806 | DPH4 intron 2 |
| 20 | rs578666 | G/A | 31361060 | 6342 | 0.383721 | DPH4 intron 2 |
| 21 | rs6484503 | G/T | 31381179 | 20119 | 0.32 | DPH4 intron 2 |
| 22 | rs1223118 | G/C | 31427076 | 45897 | 0.00384615 | IMMP1L intron 5 |
| 23 | rs1223068 | G/T | 31436925 | 9849 | 0.25 | IMMP1L intron 4 |
| 24 | rs1223098 | T/G | 31463483 | 26558 | 0.472222 | IMMP1L intron 1 |
| 25 | rs509628 | C/T | 31491931 | 28448 | 0.480159 | ELP4 intron 1 |
| 26 | rs502794 | C/A | 31503803 | 11872 | 0.484375 | ELP4 intron 2 |
| 27 | rs2996470 | T/C | 31516234 | 12431 | 0.247826 | ELP4 intron 2 |
| 28 | rs2973127 | C/T | 31519594 | 3360 | 0.251908 | ELP4 intron 3 |
| 29 | rs2104246 | G/A | 31530222 | 10628 | 0.246094 | ELP4 intron 3 |
| 30 | rs2996464 | C/T | 31545775 | 15553 | 0.265385 | ELP4 intron 3 |
| 31 | rs2146569 | G/T | 31565684 | 19909 | 0.244186 | ELP4 intron 3 |
| 32 | rs10835793 | T/A | 31575426 | 9742 | 0.25 | ELP4 intron 4 |
| 33 | rs1232182 | A/T | 31589144 | 13718 | 0.267176 | ELP4 intron 5 |
| 34 | rs986527 | T/C | 31593057 | 3913 | 0.425197 | ELP4 intron 5 |
| 35 | rs11031434 | A/G | 31609788 | 16731 | 0.492308 | ELP4 intron 6 |
| 36 | rs1232203 | A/C | 31622784 | 12996 | 0.25 | ELP4 intron 7 |
| 37 | rs964112 | T/G | 31635524 | 12740 | 0.414634 | ELP4 intron 9 |
| 38 | rs2862801 | A/G | 31652912 | 17388 | 0.248062 | ELP4 intron 9 |
| 39 | rs10835810 | T/C | 31679060 | 26148 | 0.425532 | ELP4 intron 9 |
| 40 | rs12365798 | C/T | 31704334 | 25274 | 0.244275 | ELP4 intron 9 |
| 41 | rs2863231 | A/G | 31753136 | 48802 | 0.380952 | ELP4 intron 9 |
| 42 | rs3026411 | A/T | 31758120 | 4984 | 0.334615 | ELP4 intron 9 |
| 43 | rs1506 | A/T | 31766874 | 8754 | 0.223077 | PAX6 3′ |
| 44 | rs2239789 | A/T | 31772472 | 5598 | 0.480769 | PAX6 intron 8 |

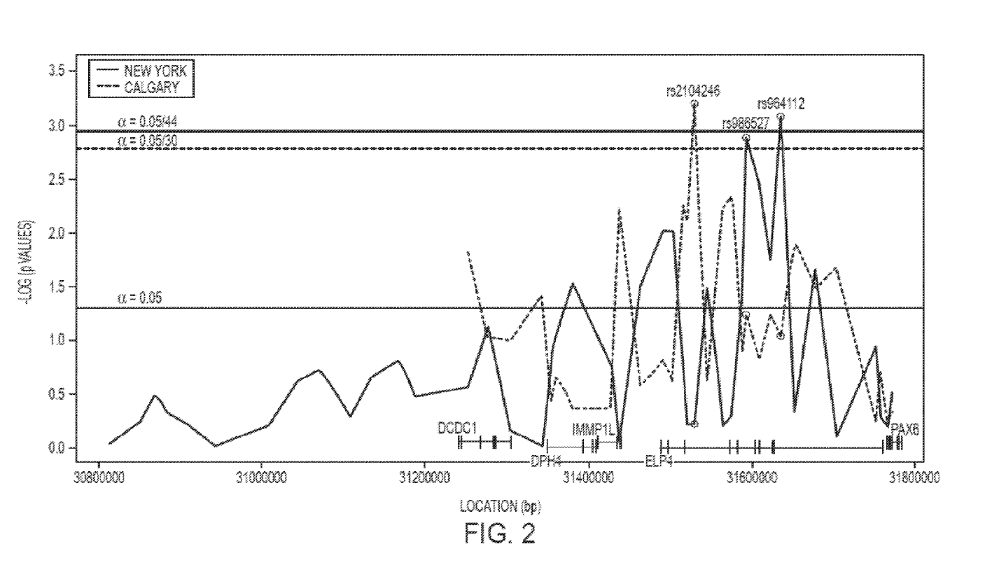

SNP markers. In the first stage polymorphic SNP markers in the 11p13 linkage region were typed, delimited by a LOD score of 1.0 on either side of the multipoint linkage peak. 36 markers were distributed predominantly within known genes, using Tagger implemented in Haploview [26] using a r²=0.8; then eight additional SNPs were typed in the region of ELP4 and PAX6 where there was evidence of association. The 44 markers were placed in and between ESTs and genes annotated in Ensemble Release 46, from downstream to upstream (see FIG. 2): DCDC5, DCDC1, DPH4, IMMP1L, ELP4, and PAX6 between 30,819,214 to 31,780,205 base pairs (hereafter “bp”) (NCBI Build 36 coordinates). In the second stage involving the replication dataset, a subset of 30 SNPs spanning the region 31,252,249 to 31,772,472 bp were typed.

SNP genotyping. DNA samples were genotyped on the Nanogen platform at deCODE Genetics (Iceland). SNPs were analyzed by end point scatter plot analysis utilizing the ABI 799HT Sequence Detection System. Sixty-eight cases, parents of 30 of these cases, and 187 controls were typed from the discovery set; all 38 cases and 138 controls were typed from the replication set. Only one SNP, rs10835810 had >5% missingness (30% missing rate, similar in cases and controls), and only rs2863231 was out of Hardy-Weinberg equilibrium in controls at the 0.001 level. All except one SNP (rs1223118) had a minor allele frequency >0.15 (Table 3).

Resequencing. PCR reactions (20 μL), consisting of ˜50 ng DNA, 1 μM forward and reverse primers, 500 μM deoxynucleotide triphosphates, 0.5 U AccuTaq LA polymerase, and 1× AccuTaq buffer (Sigma, D-1938), were carried out as follows: 3 min denaturation at 95° C., 30 cycles of PCR (95° C. denaturation, 30 sec; 57° C. annealing, 15 sec; 72° C. extension, 2 min 30 sec) and a final 10 min extension at 72° C. Reaction cleanup consisted of incubation for 15 min at 37° C. with exonuclease I and shrimp alkaline phosphatase (ExoSapIT kits, USB P/N 78201, using half the recommended amount of enzymes), followed by 15 min at 80° C. Sequencing reactions were conducted on ˜10% of the cleaned up products in 20 μA volumes, and included 1/20 reaction volume Big Dye Terminator sequencing cocktail version 3.1 diluted with recommended sequencing buffer (ABI) and 1 μM forward or reverse primer. Sequencing reactions were carried out using the following temperature profile for 35 cycles: 96° C. denaturing, 10 sec; 50° C. annealing, 5 sec; 60° C. extension, 2 min 30 sec. Sequencing products were precipitated with 0.3M sodium acetate, 70% ethanol at −20° C. for 20 min; the precipitates were pelleted, washed with 70% ethanol, and dissolved in 10 μA 100% formamide, heated for 10 min at 96° C., and analyzed using an ABI 3730×1 sequencer. Traces were examined individually, or the Seqman program (DNAStar) was used to align sequences and call homozygous variants and heterozygotes

Generally, the nomenclature and terminology used in connection with the described techniques of molecular genetics, molecular biology, and genetics described herein are those well known and commonly used in the art, as described in various general and more specific references such as those that are cited and discussed throughout the present specification.

### CTS Links to Markers at 11p13

Only markers on chromosome 11 yielded two-point genome wide LOD scores exceeding 3.0. Markers in the region of chromosomal band 11p13 provided strong and compelling evidence for linkage to CTS. Marker D11S4102 yielded a two-point LOD score of 4.01, and seven other markers in the immediate region also exhibited LOD scores exceeding 2. Both European and non-European ancestry families contributed proportionally to the LOD score. The markers on chromosome 11 generally maximized at unequal male-female recombination fractions, because the male-female recombination map differs substantially in this region. For example, at D11S4102 the recombination rate for females is 1.70 cM/MB, while for males it is 0.48 cM/MB. Two point LOD score maximization in this region of 11p most often occurred at 95% penetrance. Although single markers on chromosomes 5, 9, 10, 12 and 16 provided two-point LOD scores >2.0, the flanking marker information was not generally compelling. We did not observe significant evidence of linkage at markers previously reported for CTS at 15q14 [17] (D15S165—maximum LOD score 0.1381); nor for a rare recessive variant of RE at 16p12-11.2 [13] (D16S3068—max LOD score 0.2959), nor for X-linked rolandic seizures and cognitive deficit (MIM 300643) [14] (DXS8020—max LOD score 0.39). Similarly, evidence of linkage to 11p13 in an autosomal dominant variant of RE with speech dyspraxia and cognitive impairment [15] was not found.

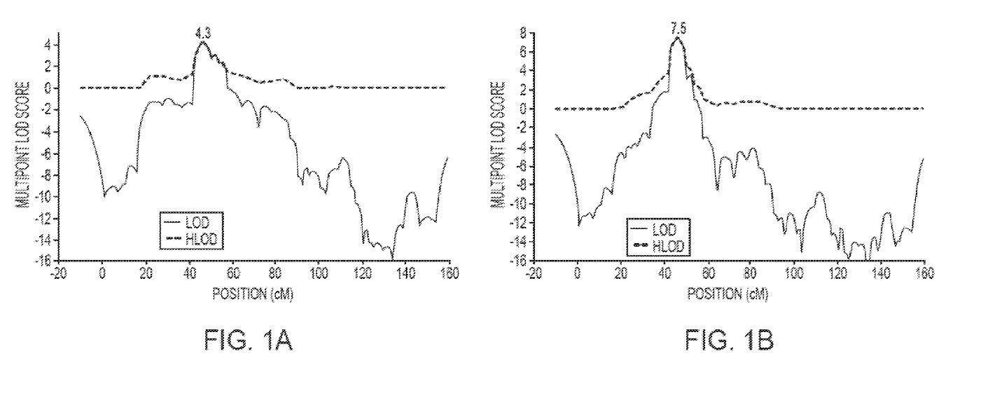

FIG. 1A shows the heterogeneity (“HLOD”) and homogeneity (“LOD”) linkage results observed in the multipoint analysis of chromosome 11, for a dominant mode of inheritance with 50% penetrance. This analysis model resulted in the highest multipoint LOD scores: 4.30 at marker D11S914 (7.4 cM from the two-point maximum). There was no showing of heterogeneity (\hat{α}=1) in the region of linkage. The region bounded by LOD scores >2.0 spans from 43.17 cM-56.88 cM, with D11S914 located at 46.7 cM [33], and includes the following annotated genes: DCDC5, DCDC1, DPH4, IMMP1L, ELP4, PAX6.

Association of CTS with SNPs in ELP4

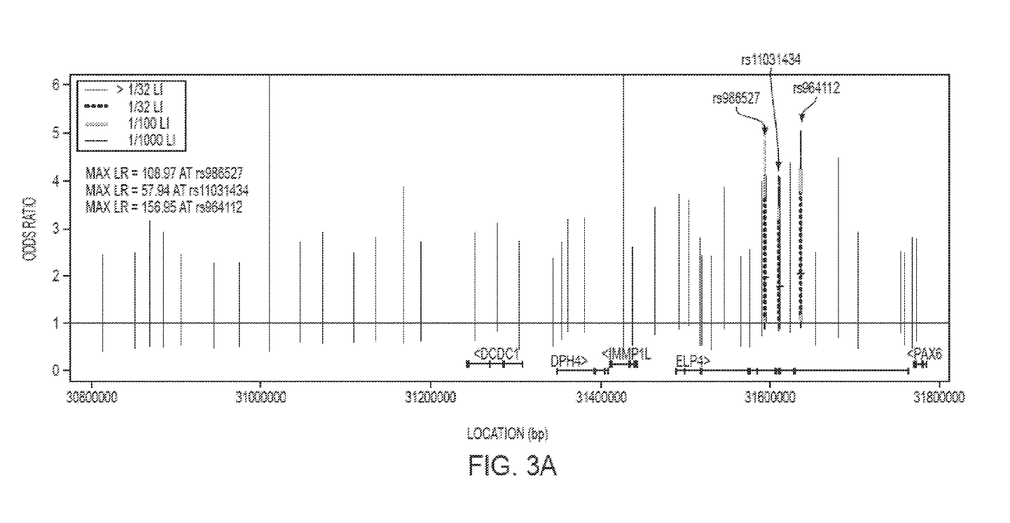

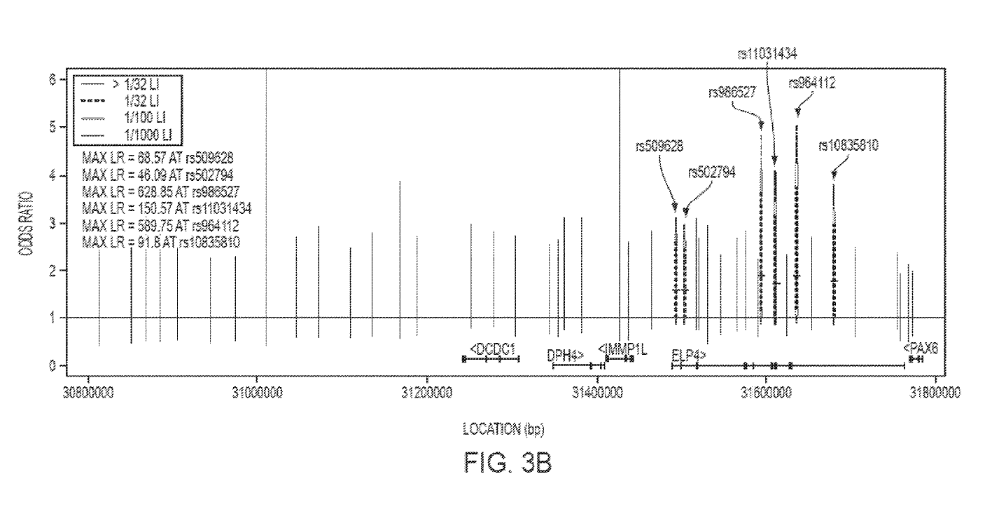

A total of 44 SNPs across the linkage region in 68 cases and 187 controls (discovery set). Here, a pure likelihood analysis was conducted, as well as computing standard Cochran-Armitage trend test p-values for comparison. The pure likelihood analysis is particularly well-suited to a joint analysis of discovery and replication samples [27], and has been noted to be particularly appropriate for genetic data [20,34]. The pure likelihood analysis plots odds ratio (OR) on the y-axis versus base pair position on the x-axis. Evidence for association at a given SNP is determined by calculating the likelihood ratio (LR); whether a calculated LR provides strong association evidence is interpreted via LOD score benchmarks: for example, LOD>1.5 (equivalent to a LR>32) is interpreted as reasonably strong association evidence. We found no evidence of association with SNPs in DCDC5, DCDC1, DPH4, IMMP1L or PAX6 as indicated by grey LIs on FIG. 3. The longer grey lines indicate lack of information, mainly due to low minor allele frequency. However, significant evidence of association with SNPs in ELP4 with both the Cochran-Armitage trend test and the pure likelihood analysis (see colored LIs in FIG. 3) was not found. FIGS. 2 and 3, Table 4 for summary statistics) with estimated ORs 1.80-2.04 at these markers. We ensured that all SNPs that had r²>0.8 with rs964112 were genotyped, but none were identified as functionally significant. In the family-based p-value analysis using FBAT, only SNPs in ELP4 provided evidence of association, with the smallest p-values observed at rs986527 (p=0.06) and rs1232182 (p=0.04) with 27 and 28 informative families, respectively. These results argue against population stratification as a positive confounder for the observed ELP4 association. Rs1232182 (p=0.04) is in complete linkage disequilibrium (i.e. not an independent determinant) with the other markers in ELP4.

TABLE 4
SINGLE SNP ASSOCIATION RESULTS: PURE LIKELIHOOD
AND FREQUENTIST ANALYSES AT SNPS OF INTEREST IN ELP4;
P-VALUES ARE UNADJUSTED

| | | Discovery analysis | | | | Joint analysis | | | |
|---|---|---|---|---|---|---|---|---|---|
| | Risk | | | Max | | | | Max | |
| SNP | allele | OR | 1/32 LI | LR | P | OR | 1/32 LI | LR | P |
| rs964112 | G | 2.04 | 1.15-3.80 | 156.95 | 0.0008 | 1.88 | 1.18-3.06 | 589.75 | 0.0002 |
| rs11031434 | G | 1.80 | 1.05-3.16 | 57.94 | 0.0035 | 1.71 | 1.10-2.70 | 150.57 | 0.0013 |
| rs986527 | C | 1.98 | 1.12-3.66 | 108.97 | 0.0013 | 1.88 | 1.18-3.06 | 628.85 | 0.0002 |

FIG. 2 shows the observed −log 10 p-values plotted for the discovery and replication samples, with a horizontal line indicating the Bonferroni critical values for the replication (α=0.05/30), and discovery (α=0.05/44) samples. Pure likelihood analysis provides a mechanism for joint analysis of discovery and replication datasets. In the pure likelihood joint analysis, the replication sample confirms that SNPs in ELP4 are highly associated with CTS; however, when analyzed on its own (FIG. 2) in a standard p-value analysis, only rs2104246 passes a Bonferroni criterion (p=0.0006). Although rs2104246 was not one of the SNPs of interest from the discovery set, it is in high LD with SNPs of interest from the discovery set. FIG. 3 depicts the pure likelihood analysis for the combined sample. Here, the association evidence for all three SNPs of interest from the discovery set has increased after combination with the replication dataset. The maximum LR at rs964112 is now 589.75 (formerly 156.95 in the discovery set), which is evidence equivalent to observing a LOD score of 2.77; and at rs986527 the maximum LR=628.85 (LOD equivalent of 2.80). The estimated ORs represent a 2-fold increase in risk of CTS. The ORs, 1/32 LIs, maximum LRs and trend test p-values from the discovery and joint analyses are displayed in Table 4. We have reported analysis of combined ancestry data, although the results are qualitatively similar when restricted to European ancestry data. The substantial increase in maximum LR from joint analysis of the two datasets provides compelling evidence that the ELP4 variants, specifically rs964112s in intron 9, rs1232182 in intron 5 and rs986527 in intron 5, are indeed associated with CTS in RE families.

### Multi-SNP Analysis

We used multiple logistic regression for multi-SNP analysis [35]. We also constructed haplotypes in Phase 2.1.1 [30, 36] and tested for differences between cases and controls in the frequency of haplotypes and haplogenotypes. The D′ from Haploview, was calculated from the European ancestry controls in the discovery dataset. A haploview plot of the linkage disequilibrium at the 11p13 locus, as measured by D′ revealed four distinct LD blocks: Block 1 spans markers in DCDC5; Block 2 spans markers between DCDC5 and DCDC1; Block 3 spans markers in DCDC1, DPH4 and IMMP1L; and Block 4 spans markers in IMMP1L. The SNPs of interest are in high LD with each other, which indicates that it is less likely that multiple independent variants in the region of ELP4 were detected. Multiple logistic regression analysis indicated that rs964112 was the best predictor of CTS, with no other SNP main effects or two-way interactions significant in the model; in the absence of rs964112, rs986527 played a similar predictive role. These SNPs were almost completely correlated. Consequently, haplotype analysis did not produce a haplotype or haplo-genotype associated with greater CTS risk than that estimated with rs964112 or rs986527 alone (Table 4).

### Resequencing Coding Regions of ELP4

We resequenced the coding portions, exon-intron boundaries and 5′ upstream region of the ELP4 gene in 40 RE probands from the discovery set. The 274 kb ELP4 gene is transcribed into a 1584 bp mRNA consisting of 12 exons, a 35 bp 5′ UTR and a 257 bp 3′ UTR. Alternative transcripts have been reported that include or exclude the last two exons. Primers were designed for direct sequencing of each of these 12 exons including some adjacent intronic sequence, as well as the putative promoter region; a list of these primers is included in Table 5. The same primers were used for PCR and sequencing reactions. After alignment, all homozygous and heterozygous variants within the sequenced region were noted.

Three previously reported SNP variants were found in these 40 individuals: rs2295748 in the vicinity of the promoter; rs2273943 within intron 5 located 127 bases upstream of exon 6, and rs10767903, located within exon 10. The genotypes and allele frequencies for these SNPs in these individuals were compared with those available through dbSNP. The minor allele for rs2295748 was slightly less common in the 40 RE cases (0.22) than in any of the AFD or CEPH populations, while the minor allele for rs2273943 occurred in these cases at approximately the same frequency (0.24) as in the Caucasian and Chinese CEPH populations. Frequency information was not available for comparison for rs10767903 so 85 controls at this SNP were typed. The T allele at rs10767903 is predicted to abolish an adjacent splice donor enhancer site that would result in skipping of alternative exons 10 and 11. Out of 36 RE probands that were typed at this synonymous polymorphism, 34 carried the T allele: 21 TT, 13 CT, 2CC. However, controls exhibited a similar genotypic distribution: 42 TT, 34CT, 9CC.

TABLE 5
PCR AND SEQUENCING PRIMERS. ALL PRIMERS HAVE SIMILAR
SALT ADJUSTED MELTING TEMPERATURES (RANGE 63-64° C.)

| | | | Product |
|---|---|---|---|
| Position | Forward Primer (5′-3′) | Reverse Primer (5′-3′) | Length |
| Promoter | AGAGATCCCATCCTTTCCATATA | ACCCGTCCTATCAGAACCAGTG | 466 |
| | AC (SEQ ID NO: 1) | (SEQ ID NO: 2) | |
| Exon 1 | ACGTCTCAGTCCTATTGGTTACG | CTCCCTAAGTTTCCCCTCGG | 399 |
| | (SEQ ID NO: 3) | (SEQ ID NO: 4) | |
| Exon 2 | ACTACTGTTTTAAAGTTATTGAA | AGAGCTACATGTTCAGATATATT | 358 |
| | GTGCC (SEQ ID NO: 5) | TGCC (SEQ ID NO: 6) | |
| Exon 3 | TGAGTGTGCTTGCTGTTTGATAG | TGGTTCCGTTAATGCATTTAAAT | 323 |
| | C (SEQ ID NO: 7) | ATAGTTTG (SEQ ID NO: 8) | |
| Exon 4 | TCAATGTTAGTCATGAATTTTCA | ACATATAGGCATACCACAAGAG | 336 |
| | ATACATTG (SEQ ID NO: 9) | ATTC (SEQ ID NO: 10) | |
| Exon 5 | TGCCATTGTTTTGCTGGATGTAG | TGATATTTACCCTTAGATGTGTAT | 327 |
| | G (SEQ ID NO: 11) | TCTTTC (SEQ ID NO: 12) | |
| Exon 6 | AGGAACACTGAGCAAGTTATAA | ACTTCTGGGTTCCCGCCCC | 382 |
| | TAAGG (SEQ ID NO: 13) | (SEQ ID NO: 14) | |
| Exon 7 | AACACATCTATTGACATTGTCTC | AGATGGTCAACATCATTAGTTAT | 408 |
| | CC (SEQ ID NO: 15) | CATGG (SEQ ID NO: 16) | |
| Exon 8 | TGTTGATAGTCTATCTCCACTAC | AGCTGCCATGGAAGACTGGAC | 380 |
| | AG (SEQ ID NO: 17) | (SEQ ID NO: 18) | |
| Exon 9 | AGGATGCTTGTGTGTAAATTTAC | CATAAAACATGTCCTAAGAATTT | 339 |
| | AGG (SEQ ID NO: 19) | CATTAAAG (SEQ ID NO: 20) | |
| Exon 9a | ACTGATAGGTGCTTGAACAAAC | AGCTTGGCTGAAACTGTTGCATA | 455 |
| | AGG (SEQ ID NO: 21) | G (SEQ ID NO: 22) | |
| Exon 9b | CCTTTCCTGTCGCTTGATTTGTTG | AGCAGTATGTGAACACCTTAAAC | 425 |
| | (SEQ ID NO: 23) | TATC (SEQ ID NO: 24) | |
| Exon 10 L | TGTAATCTGAAGTATGCTAGCCA | TGTTTTTCAAGGAGTGGAGGGTC | 332 |
| | AAG (SEQ ID NO: 25) | (SEQ ID NO: 26) | |
| Exon 10 R | AGGGATTCCTCCTTAGTCGCTG | TGTATGCTACCTGCTGTGACATG | 340 |
| | (SEQ ID NO: 27) | (SEQ ID NO: 28) | |

### Rare Mendelian Forms

Autosomal Dominant with Speech Dyspraxia and MR (Scheffer, 1995)

Autosomal Dominant with Dyspraxia and MR (Kugler, 2007)

Autosomal Recessive with Dystonia 16p12 (Guerrini, 1999)

X-linked with MR (Roll, 2006)

There are several reasons why these results are unlikely to be spurious. The localization of ELP4 was conducted through genome wide linkage analysis: only one area of the genome at 11p13 showed strong and compelling evidence for linkage to CTS. Under that linkage peak, fine-mapping evidence unambiguously pointed to the association of CTS with SNP markers in ELP4. SNPs in ELP4 were associated with increased risk of CTS in both discovery and replication datasets, with evidence for association of the same SNPs in each dataset. Furthermore, not only the same SNPs but the same alleles were associated with increased risk of CTS in both datasets. Interestingly, no evidence of locus or allelic heterogeneity based on ancestry was found in either linkage or association analyses. In addition, the association in the discovery set was confirmed using FBAT, which mitigates concerns about positive confounding due to population stratification.

The mapping of CTS to ELP4 shows that the common form of RE and rare variants of RE are genetically heterogeneous. Our data revealed little or no evidence of linkage to recessive (MIM 608105) [13] or X-linked (MIM 300643) [14] variants of rolandic epilepsy; neither did a rare autosomal dominant form of RE with speech dyspraxia and cognitive impairment show linkage to 11p13 [15]. Thus it seems that loci in Mendelian variants of RE may represent “private” mutations.

Although an association with SNPs across ELP4 was found, regression analysis indicated that spread of association evidence could be explained by linkage disequilibrium around rs986527 in intron 9, LD which stretches to IMMP1L and the 3′ end of ELP4, but not to PAX6. Subsequent resequencing of the coding, boundary and promoter regions revealed no enrichment of ELP4 exonic polymorphisms among probands. Exclusion of the coding sequences shows that the genetic effector may lie in the non-coding regions of ELP4. It is less likely that the causative mutation lies in a distant gene beyond IMMP1L upstream or PAX6 downstream because of the drop-off in linkage disequilibrium at subjacent markers.

Substantiating ELP4 as a high risk locus for CTS is an important step in assembling the complex genetic model of RE. Without being bound by theory, we hypothesize that an as yet unidentified non-coding mutation exists that is in linkage disequilibrium with SNPs in ELP4 intron 9. This hypothesized mutation impairs brain-specific Elongator function during brain development, possibly mediated via interaction with genes and proteins in cell migration and actin cytoskeleton pathways. Additional genetic factors though, may need to be invoked to explain the occurrence of seizures and reading disability in RE. For example, while CTS is common in children [6], only an estimated 10% of children with the trait manifest clinical seizures [11]. At the same time, there is no evidence for an environmental contribution to RE. Thus, while CTS is mandatory for the definition of RE, additional genetic factors, which likely act in combination with the ELP4 locus to cause the classic focal seizures of RE, remain to be elucidated.

URLs. Mendelian variants of RE are listed in Online Inheritance in Man http://www.ncbi.nlm.gov/entrez. The Haploview application can be downloaded at http://www.broad.mit.edu/mpg/haploview. Information about marker location can be found at UniSTS at the NCBI website above, and through the Ensemble genome browser at http://www.ensembl.org/Homo_sapiens/index.html. SNP frequencies were accessed at dbSNP: http://www.ncbi.nlm.nih.gov/projects/SNP. The ESE Finder program was used to assess alternative exon 10 of ELP4 and can be accessed at http://rulai.cshl.edu/cgi-bin/tools/ESE3/esefinder.cgi?process=home. For bioinformatic analyses of ELP4 protein variants produced by alternative gene splicing PHI-BLAST was used, and accessed PANTHER and Bioinformatics websites as follows: http://www.pantherdb.org and http://bioinformatics.weizmann.ac.il/blocks/blockmkr/www/make_blocks.html.

### Example 2

### Speech Dyspraxia is the Neural Mechanism for Speech Sound Disorder in RE Families

Subjects: Probands were enrolled if they met stringent eligibility criteria for RE, including typical orofacial seizures, age of onset between 3-12 years, no previous epilepsy type, normal global developmental milestones, normal neurological examination, EEG with centrotemporal sharp waves (CTS) and normal background, and neuroimaging that excluded an alternative structural, inflammatory or metabolic cause for the seizures (62). Neuroimaging was reviewed by readers blinded to the identity and diagnosis of the subjects (63).

Measures: Siblings, parents and grandparents were directly interviewed by a physician, using a 125 item questionnaire (3), to ascertain the clinical history of the child, siblings and both parents. As well as a perinatal, developmental and school history for children, the presence of SSD symptoms were elicited using operational ICD-10 definitions of speech articulation disorder (F80.0) (who.int/classifications/en) (Tunick R A, Pennington B F. The etiological relationship between reading disability and phonological disorder Annals of Dyslexia. 2002; 52:75-97.); a similar operational definition of SSD has been used in a high risk study of phonological disorder. History based assessments of a lifetime history of SSD are more reliable than clinical examination, because SSD has often resolved by middle to late childhood. The same questionnaire items were used, with minor modifications for age, for the probands, siblings and parents. Siblings between the ages of 4-16 years underwent sleep-deprived EEGs to assess their CTS status (16). CTS is an autosomal dominant, age-dependent trait that disappears after the age of 16 years. We coded individuals as unknown if they were above 16 years, or if they had no CTS on a wake EEG and did not have sleep EEG. We assessed the speech recordings of 18 consecutively recruited probands (all CTS positive by definition), their 16 siblings aged 6 years and above, and 15 available parents of probands. Blood or saliva samples (ORAGENE) for DNA extraction were collected from probands and all potentially informative available family members. The study was approved by the IRBs of New York State Psychiatric Institute and all collaborating centers. Subjects gave written informed consent.

Speech recording, sampling and analysis: The subjects each read a list of 40 English monosyllabic words such as big, keep, dig, beginning and ending with the stop consonants [b], [p], [d], [t], [g] and [k]. The word list was repeated twice, and digitally recorded in a quiet location using an electret condenser microphone. The tape recordings were then sampled at a rate of 20,000 samples per second with 16 bit quantization using the interactive BLISS speech analysis system (64). The resulting bandwidth was 10 kHz bandwidth, which preserves the salient acoustic cues for the perception of adult speech (65).

The digitized speech signal was then segmented into the individual words using the BLISS system's waveform display which allows an operator to place cursors to delimit and listen to any segment of the speech signal. Four independent sets of cursors can be placed on a waveform to delimit and measure intervals; the operator can increase the resolution of the time base or the amplitude of the displayed speech signal. Discrete Fourier transforms that yield frequency analyses of the signal in any segment can be produced as well as estimates of the formant frequencies of the speech signal (64). Stop consonants are produced by first obstructing the airway above the larynx with the lips or tongue. A sudden “burst” of acoustic energy occurs when the occlusion is released and is followed by periodic phonation produced by the vocal folds of the larynx. Voice-onset time (VOT) is the interval between the initial burst and the onset of phonation. It is a key acoustic cue that differentiates the “voiced” stop consonants [b], [d] and [g] from their “unvoiced” counterparts [p], [t], and [k] in English and many other languages (66). Voiced consonants are characterized by a VOT of less than 25 msec, while voiceless consonants have a VOT of greater than 25 msec. Speakers must control the sequence between the release of the stop and the start of phonation.

VOT has proven to be highly correlated with cognitive and sentence comprehension deficits in subjects suffering insult to the basal ganglia in Parkinson's disease and hypoxia. Correct VOT production relies upon the proper temporal coordination of laryngeal and supralaryngeal motor events, adult apraxic patients demonstrate VOT overlap between normally separate, bimodal VOT distributions. Vowel duration has proven to be a predictor of cognitive dysfunction in hypoxic subjects and errors in sentence comprehension in aged, otherwise neurologically intact people.

The speech production metrics calculated in this study were: (1) average vowel duration, which provides a measure of speaking rate; (2) VOT “minimal separation”, the shortest interval differentiating a voiced stop consonant from the unvoiced stop produced by a similar articulatory maneuver. The linguistic term characterizing these different maneuvers is “place of articulation”, i.e., the English bilabial stops, [b] and [p], in which the lips occlude the vocal tract, the English alveolar stops, [d] and [t], in which the tongue blade occludes the vocal tract, and the velar stops, [g] and [k], in which the tongue body occludes the vocal tract; (3) mean VOTs for these different places of articulation.

Vowel durations and dispersion are greater for younger children than for older children or adults. We therefore compared our results with normative vowel duration data from our own laboratory and from a sample of 436 children ages 5 through 18 years and 56 adults ages 25 to 50 years for ten consonant-vowel-consonant words produced in sentence frame or in isolation (71). VOT ranges were compared with normative data for adults (66) and children (72). VOT overlap and convergence occur when the ranges of VOT for stop consonants such as [b] versus [p] overlap or fall below 20 msec. VOT dispersion is evident in variance beyond the normal range for the “unvoiced” stop consonants [p], [t] and [k]. VOT metrics were calculated for subjects blind to their identity, seizure, EEG or developmental history. Vowel durations were coded as normal range (−), beyond normal range (+) or abnormal (++). VOT were coded as normal range (−), overdispersed (+), or overlapping (++), Table 6

TABLE 6

| | | | |
|---|---|---|---|
| | | Voice Onset | Vowel |
| Sex | Age | Time | Duration |

Probands

| | | | |
|---|---|---|---|
| M | 12 | ++ | ++ |
| M | 16 | ++ | ++ |
| M | 7 | ++ | ++ |
| M | 12 | ++ | + |
| M | 12 | + | ++ |
| M | 8 | + | ++ |
| M | 9 | + | ++ |
| M | 12 | + | − |
| M | 9 | − | ++ |
| M | 8 | − | − |
| M | 10 | − | − |
| M | 9 | − | − |
| F | 11 | ++ | ++ |
| F | 11 | + | ++ |
| F | 12 | − | ++ |
| F | 13 | − | ++ |
| F | 11 | − | − |
| F | 8 | − | − |

Siblings

| | | | |
|---|---|---|---|
| M | 10 | ++ | ++ |
| M | 6 | ++ | ++ |
| M | 9 | ++ | ++ |
| M | 13 | ++ | ++ |
| F | 8 | ++ | ++ |
| F | 10 | ++ | + |
| F | 22 | + | ++ |
| F | 10 | + | − |
| F | 15 | − | ++ |
| F | 12 | − | ++ |
| F | 5 | − | ++ |
| F | 14 | − | ++ |
| F | 14 | − | ++ |
| F | 18 | − | + |
| F | 14 | − | − |
| F | 10 | − | − |

Parents

| | | | |
|---|---|---|---|
| M | 48 | ++ | ++ |
| M | 35 | + | ++ |
| M | 50 | − | ++ |
| M | 44 | − | − |
| M | 38 | − | − |
| M | 42 | − | − |
| F | 48 | ++ | ++ |
| F | 38 | + | ++ |
| F | 35 | + | ++ |
| F | 53 | − | ++ |
| F | 41 | − | ++ |
| F | 44 | − | − |
| F | 38 | − | − |
| F | 47 | − | − |
| F | 43 | − | − |

### Table 4. Praxic Errors

VOT: normal (−) dispersion (+) overlap (++);
Vowel duration, VD normal (−) high normal (+) lengthened (++)

Genotyping: A total of 194 individuals were genotyped using the genomewide deCODE 1000 marker single tandem repeat (STR) panel, which has an average genome-wide resolution of 4 cM. Amplified fragments were typed using ABI 3700 and ABI 3730 DNA analyzers with CEPH family DNA used as standards. Alleles were called automatically and checked for consistency with Hardy-Weinberg equilibrium and Mendelian consistency. Genotype data were then integrated with affectedness and pedigree data.

Linkage Analysis: We analyzed the data by two-point and multipoint lod score calculations using the Maximized Maximum Lod Score (MMLS) approach (73): lod scores were calculated under both dominant and recessive modes of inheritance. We specified a dominant gene frequency of 0.006, a recessive gene frequency at 0.1, a sporadic rate at 0.002, and penetrance of 0.50. In regions providing significant evidence for linkage, we then maximized over penetrance. Marker allele frequencies were calculated from the dataset. We then followed up those two-point results that provided lod scores greater than 3.0 with multipoint analysis using Genehunter (74), again using the MMLS approach followed by penetrance maximization and computation of heterogeneity lod scores. For more discussion on statistical genetic methods, see Strug (2009). FIG. 1B shows SSD/EEG; dominant model with 50% penetrance, max at D11S914 (48 cM).

Table 7. Maximum single point lodscores observed on chromosome 11 for all three phenotypes; dominant model.

TABLE 7

| | | Maxlod | | Number of |
|---|---|---|---|---|
| Phenotype | Marker | (flanking) | cM (flanking) | families |
| SSD | D11S2368 | 2.30 | 29.3 | 28 |
| | | (1.30, 1.37) | (26.2, 34.9) | |
| EEG | D11S4102 | 4.01 | 55.4 | 37 |
| | | (1.58, 2.03) | (52.0, 58.1) | |
| SSD/EEG | D11S4102 | 4.61 | 55.4 | 37 |
| | | (2.71, 2.61) | (52.0, 58.1) | |

The invention is illustrated herein by the experiments described above and by the following examples, which should not be construed as limiting. The contents of all references, pending patent applications and published patents, cited throughout this application are hereby expressly incorporated by reference. Those skilled in the art will understand that this invention may be embodied in many different forms and should not be construed as limited to the embodiments set forth herein. Rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

### REFERENCES

1 Shinnar S, O'Dell C, Berg A T (1999) Distribution of epilepsy syndromes in a cohort of children prospectively monitored from the time of their first unprovoked seizure. Epilepsia 40: 1378-1383
2. Beaussart M (1972) Benign epilepsy of children with Rolandic (centro-temporal) paroxysmal foci: a clinical entity. Study of 221 cases. Epilepsia 13: 795-811.
3. Clarke T, Strug L J, Murphy P L, Bali B, Carvalho J, et al. (2007) High risk of reading disability and speech sound disorder in rolandic epilepsy families: case-control study. Epilepsia 48: 2258-2265.
4. Kavros P M, Clarke T, Strug L J, Halperin J M, Dorta N J, et al. (2008) Attention impairment in rolandic epilepsy: Systematic review. Epilepsia: April 11. Epub ahead of print.
5. Boxerman J, Hawash K, Bali B, Clarke T, Rogg J, et al. (2007) Is Rolandic epilepsy associated with abnormalities on cranial MRI? Res 75: 180-185.
6. Eeg-Olofsson O, Petersen I, Sellden U (1971) The development of the electroencephalogram in normal children from the age of 1 through 15 years. Paroxysmal activity. Neuropadiatrie 2: 375-404.
7. Echenne B, Cheminal R, Rivier F, Negre C, Touchon J, et al. (1992) Epileptic electroencephalographic abnormalities and developmental dysphasias: a study of 32 patients. Brain Dev 14: 216-225.
8. Holtmann M, Becker K, Kentner-Figura B, Schmidt M H (2003) Increased frequency of rolandic spikes in ADHD children. Epilepsia 44: 1241-1244.
9. Scabar A, Devescovi R, Blason L, Bravar L, Carrozzi M (2006) Comorbidity of DCD and SLI: Significance of epileptiform activity during sleep. Child Care Health Dev 32: 733-739.
10. Doose H, Neubauer B, Carlsson G (1996) Children with benign focal sharp waves in the EEG—developmental disorders and epilepsy. Neuropediatrics 27: 227-241.
11. Heijbel J, Blom S, Rasmuson M (1975) Benign epilepsy of childhood with centrotemporal EEG foci: a genetic study. Epilepsia 16: 285-293.
12. Scheffer I E, Jones L, Pozzebon M, Howell R A, Saling M M, et al. (1995) Autosomal dominant rolandic epilepsy and speech dyspraxia: a new syndrome with anticipation. Ann Neurol 38: 633-642.
13. Guerrini R, Bonanni P, Nardocci N, Parmegianni L, Piccirilli M, et al. (1999) Autosomal recessive Rolandic epilepsy with paroxysmal exercise-induced dystonia and writer's cramp: delineation of the syndrome and gene mapping to chromosome 16p12-11.2. Ann Neurol 45: 344-352.
14. Roll P, Rudolf G, Pereira S, Royer B, Scheffer I E, et al. (2006) SRPX2 mutations in disorders of language cortex and cognition. Hum Mol Genet 15: 1195-1207.
15. Kugler S L, Bali B, Lieberman P, Strug L J, Gagnon B R, et al. (2007) An Autosomal Dominant Genetically Heterogeneous Variant of Rolandic Epilepsy Epilepsia doi:10.1111/j.1528-1167.2007.01517.x.
16. Bali B, Kull L, Strug L, Clarke T, Murphy P L, et al. (2007) Autosomal dominant inheritance of centrotemporal sharp waves in rolandic epilepsy families. Epilepsia 48: 2266-2272.
17. Neubauer B A, Fiedler B, Himmelein B, Kampfer F, Lassker U, et al. (1998) Centrotemporal spikes in families with rolandic epilepsy: linkage to chromosome 15q14. Neurology 51: 1608-1612.
18. Royall R M (1997) Statistical Evidence: A Likelihood Paradigm. London: Chapman and Hall.
19. Blume J D (2002) Tutorial in Biostatistics: Likelihood methods for measuring statistical evidence. Statistics in Medicine 21: 2563-2599.
20. Strug L J, Hodge S E (2006) An alternative foundation for the planning and evaluation of linkage analysis. I. Decoupling “error probabilities” from “measures of evidence”. Hum Hered 61: 166-188
21. Strug L J, Rohde C A, Corey P N (2007) An introduction to evidential sample size calculations. American Statistician 61: 207-212.
22. Commission on Classification and Terminology of the International League Against Epilepsy (1989) Proposal for revised classification of epilepsies and epileptic syndromes. Epilepsia 30: 389-399.
23. Hodge S E, Abreu P, Greenberg D A (1997) Magnitude of type I error when single-locus linkage analysis is maximized over models: a simulation study. American Journal of Human Genetics 60: 217-227.
24. Kruglyak L, Daly M J, Reeve-Daly M P, Lander E S (1996) Parametric and nonparametric linkage analysis: a unified multipoint approach. American Journal of Human Genetics 58: 1347-1363.
25. Daw E W, Thompson E A, Wijsman E M (2000) Bias in multipoint linkage analysis arising from map misspecification. Genet Epidemiol 19: 366-380.
26. Barrett J C, Fry B, Maller J, Daly M J (2005) Haploview: analysis and visualization of LD and haplotype maps. Bioinformatics 21: 263-265.
27. Strug L J, Hodge S E (2006) An alternative foundation for the planning and evaluation of linkage analysis. II. Implications for multiple test adjustments. Hum Hered 61: 200-209.
28. Benjamini Y, Hochberg Y (1995) Controlling the false discovery rate: a practical and powerful approach to multiple testing. J R Statist Soc B 57: 289-300.
29. Laird N M, Horvath S, Xu X (2000) Implementing a unified approach to family-based tests of association. Genet Epidemiol 19 Suppl 1: S36-42.
30. Stephens M, Smith N J, Donnelly P (2001) A new statistical method for haplotype reconstruction from population data. Am J Hum Genet 68: 978-989.
31. Lindsey J K (1996) Parametric Statistical Inference. Oxford: Clarendon Press.
32. Royall R M (2000) On the probability of observing misleading statistical evidence (with discussion). Journal of the American Statistical Association 95: 760-780.
33. Kong A, Gudbjartsson D F, Sainz J, Jonsdottir G M, Gudjonsson S A, et al. (2002) A high-resolution recombination map of the human genome. Nat Genet 31: 241-247.
34. Vieland V J, Hodge S E (1998) Review of Statistical Evidence: a likelihood paradigm by Royall, R. Am J Hum Genet 63: 283-289.
35. Clayton D, Chapman J, Cooper J (2004) Use of unphased multilocus genotype data in indirect association studies. Genet Epidemiol 27: 415-428.
36. Stephens M, Donnelly P (2003) A comparison of bayesian methods for haplotype reconstruction from population genotype data. Am J Hum Genet 73: 1162-1169.
37. Otero G, Fellows J, Li Y, de Bizemont T, Dirac A M, et al. (1999) Elongator, a multisubunit component of a novel RNA polymerase II holoenzyme for transcriptional elongation. Mol Cell 3: 109-118.
38. Svejstrup J Q (2007) Elongator complex: how many roles does it play? Curr Opin Cell Biol 19: 331-336.
39. Krogan N J, Greenblatt J F (2001) Characterization of a six-subunit holo-elongator complex required for the regulated expression of a group of genes in Saccharomyces cerevisiae. Mol Cell Biol 21: 8203-8212.
40. Esberg A, Huang B, Johansson M J, Bystrom A S (2006) Elevated levels of two tRNA species bypass the requirement for elongator complex in transcription and exocytosis. Mol Cell 24: 139-148.
41. Winkler G S, Kristjuhan A, Erdjument-Bromage H, Tempst P, Svejstrup J Q (2002) Elongator is a histone H3 and H4 acetyltransferase important for normal histone acetylation levels in vivo. Proc Natl Acad Sci USA 99: 3517-3522.
42. Close P, Hawkes N, Cornez I, Creppe C, Lambert C A, et al. (2006) Transcription impairment and cell migration defects in elongator-depleted cells: implication for familial dysautonomia. Mol Cell 22: 521-531.
43. Johansen L D, Naumanen T, Knudsen A, Westerlund N, Gromova I, et al. (2008) IKAP localizes to membrane ruffles with filamin A and regulates actin cytoskeleton organization and cell migration. J Cell Sci 121: 854-864.
44. Niedermeyer E, McKusick V A, Brunt P, Mahloudji M (1967) The EEG in familial dysautonomia (Riley-Day syndrome). Electroencephalogr Clin Neurophysiol 22: 473-475.
45. Anderson S L, Coli R, Daly I W, Kichula E A, Rork M J, et al. (2001) Familial dysautonomia is caused by mutations of the IKAP gene. Am J Hum Genet 68: 753-758.
46. Slaugenhaupt S A, Blumenfeld A, Gill S P, Leyne M, Mull J, et al. (2001) Tissue-specific expression of a splicing mutation in the IKBKAP gene causes familial dysautonomia. Am J Hum Genet 68: 598-605.
47. Mezey E, Parmalee A, Szalayova I, Gill S P, Cuajungco M P, et al. (2003) Of splice and men: what does the distribution of IKAP mRNA in the rat tell us about the pathogenesis of familial dysautonomia? Brain Res 983: 209-214.
48. Griffin C, Kleinjan D A, Doe B, van Heyningen V (2002) New 3′ elements control Pax6 expression in the developing pretectum, neural retina and olfactory region. Mech Dev 112: 89-100.
49. Park J I, Kim S J, Kim H G. Acoustic effects of carbamazepine in benign rolandic epilepsy. Epilepsy Behay. 2005 November; 7(3):468-71.
50. Lundberg S, Frylmark A, Eeg-Olofsson O. Children with rolandic epilepsy have abnormalities of oromotor and dichotic listening performance. Dev Med Child Neurol. 2005 September; 47(9):603-8.
51. Bladin P F. The association of benign rolandic epilepsy with migraine. In: Andermann F, Lugharesi E, editors. Migraine and Epilepsy. Boston: Butterworths; 1987. p. 145-52.
52. Doose H. Symptomatology in children with focal sharp waves of genetic origin. Eur J. Pediatr. 1989; 149(3):210-5.
53. Deonna T W, Roulet E, Fontan D, Marcoz J P. Speech and oromotor deficits of epileptic origin in benign partial epilepsy of childhood with rolandic spikes (BPERS). Relationship to the acquired aphasia-epilepsy syndrome. Neuropediatrics. 1993 April; 24(2):83-7.
54. Dubois C M, Gianella D, Chaves-Vischer V, Haenggeli C A, Deonna T, Roulet Perez E. Speech delay due to a prelinguistic regression of epileptic origin. Neuropediatrics. 2004 February; 35(1):50-3
55. Shriberg L D, et al., The speech disorders classification system (SDCS): extensions and lifespan reference data. J Speech Lang Hear Res. 1997 August; 40(4):723-40.
56. Park J I, Kim S J, Kim H G. Acoustic effects of carbamazepine in benign rolandic epilepsy. Epilepsy Behay. 2005 November; 7(3):468-71.
57. Lundberg S, Frylmark A, Eeg-Olofsson O. Children with rolandic epilepsy have abnormalities of oromotor and dichotic listening performance. Dev Med Child Neurol. 2005 September; 47(9):603-8.
58. Bladin P F. The association of benign rolandic epilepsy with migraine. In: Andermann F, Lugharesi E, editors. Migraine and Epilepsy. Boston: Butterworths; 1987. p. 145-52.
59. Doose H. Symptomatology in children with focal sharp waves of genetic origin. Eur J. Pediatr. 1989; 149(3):210-5.
60. Scheffer I E, Jones L, Pozzebon M, Howell R A, Saling M M, Berkovic S F. Autosomal dominant rolandic epilepsy and speech dyspraxia: a new syndrome with anticipation. Ann Neurol. 1995; 38(4):633-42.
61. Picard A, Cheliout Heraut F, Bouskraoui M, Lemoine M, Lacert P, Delattre J. Sleep EEG and developmental dysphasia. Dev Med Child Neurol. 1998 September; 40(9):595-9.
62. Strug U, et al. Centrotemporal sharp wave EEG trait in rolandic epilepsy maps to Elongator Protein Complex 4 (ELP4). Eur J Hum Genet. 2009 Jan 28:Epub.
63. Boxerman J, Hawash K, Bali B, Clarke T, Rogg J, Pal D K. Is Rolandic epilepsy associated with abnormalities on cranial MRI? Epilepsy Res. 2007; 75(2-3):180-5.
64. Mertus J. Brown Lab Interactive Speech System. 2005.
65. Fant G. Acoustic theory of speech production. The Hague: Mouton; 1960
66. Lisker L, Abramson AS. A cross-language study of voicing in initial stop: Acoustical measurements. Word. 1964; 20:384-442.
67. Freeman F J, Sands E S, Harris K S. Temporal coordination of phonation and articulation in a case of verbal apraxia: A voice onset time study. Brain and Language. 1978; 6:106-11.
68. Kent R D, Rosenbeck J C. Acoustic patterns of apraxia of speech. Journal of Speech and Hearing Research. 1983; 26:231-49.
69. Lieberman P, Morey A, Hochstadt J, Larson M, Mather S. Mount Everest: a space analogue for speech monitoring of cognitive deficits and stress. Aviat Space Environ Med. 2005 June; 76(6 Suppl):B198-207.
70. Lieberman P, Feldman L S, Aronson S, Engen E. Sentence comprehension, syntax and vowel duration in aged people. Clinical Linguistics and Phonetics. 1989; 3:299-311
71. Lee S, Potamianos A, Narayanan S. Acoustics of children's speech: developmental changes of temporal and spectral parameters. J Acoust Soc Am. 1999 March; 105(3):1455-68.
72. Tyler A A, Saxman J H. Initial voicing contrast acquisition in normal and phonologically disordered children. Applied Psycholinguistics. 1992; 12:453-79.
73. Hodge S E, Abreu P, Greenberg DA. Magnitude of type I error when single-locus linkage analysis is maximized over models: a simulation study. American Journal of Human Genetics. 1997; 60(1):217-27.
74. Kruglyak L, Daly M J, Reeve-Daly M P, Lander E S. Parametric and nonparametric linkage analysis: a unified multipoint approach. American Journal of Human Genetics. 1996; 58(6):1347-63.

What is claimed is:

1. A method for confirming a diagnosis of rolandic epilepsy in a patient who has had a seizure or an interictal EEG with centrotemporal sharp waves and normal background, comprising:
a. obtaining a DNA sample from the patient,
b. analyzing the DNA sample to determine if there is a nucleotide variant in the Elongator Protein Complex 4 (ELP4) gene on chromosome 11, and
c. if the nucleotide variant is detected, concluding that the patient has rolandic epilepsy.

2. The method of claim 1, wherein the gene locus is the 11p13 locus.

3. The method of claim 1, wherein the variant is an SNP that is a member of the group comprising rs964112s in intron 9, rs1232182 in intron 5 and rs986527 in intron 5 of the Elongator Protein Complex 4 gene.

4. The method of claim 1, wherein the patient has a parent or sibling with rolandic epilepsy.

5. The method of claim 1, further comprising determining if the patient has a developmental deficit that is a member selected from the group comprising a speech sound disorder, a reading disability, a developmental coordination disorder (DCD) and an attention impairment.

6. The method of claim 5, wherein the speech disorder is speech dyspraxia.

7. The method of claim 5, wherein the attention impairment is attention deficit hyperactivity disorder (ADHD).

8. The method of claim 1, wherein neuroimaging of the patient's brain excludes an alternative structural, inflammatory or metabolic cause for the seizure or the interictal EEG with centrotemporal sharp waves and normal background.

9. The method of claim 1, wherein the patient is under 15 years of age.

10. The method as in claim 1, wherein the patient's DNA sample is derived from any patient cell type, preferably cells selected from the group comprising white blood cells, saliva leukocytes, lymphoblasts, epidermal cells, and fibroblasts.

11. A method for determining that a patient has a high risk of developing rolandic epilepsy, comprising:
a. obtaining a DNA sample from the patient,
b. analyzing the DNA sample to determine if there is a nucleotide variant in the Elongator Protein Complex 4 (ELP4) gene on chromosome 11, and
c. if the nucleotide variant is detected, concluding that the patient has rolandic epilepsy.

12. The method of claim 11, wherein the gene locus is the 11p13 locus.

13. The method of claim 11, wherein the variant is an SNP that is a member of the group comprising rs964112s in intron 9, rs1232182 in intron 5 and rs986527 in intron 5 of the Elongator Protein Complex 4 gene.

14. The method of claim 11, wherein the patient has a parent or sibling with rolandic epilepsy.

15. The method of claim 11, further comprising determining if the patient has a developmental deficit that is a member selected from the group comprising a speech disorder, a reading disability, a developmental coordination disorder (DCD) and an attention impairment.

16. The method of claim 15, wherein the speech disorder is speech dyspraxia.

17. The method of claim 15, wherein the attention impairment is attention deficit hyperactivity disorder (ADHD).

18. The method of claim 11, wherein neuroimaging of the patient's brain excludes an alternative structural, inflammatory or metabolic cause for the seizure or the interictal EEG with centrotemporal sharp waves and normal background.

19. The method of claim 11, wherein the patient is under 15 years of age.

20. The method as in claim 11, wherein the patient's DNA sample is derived from any patient cell type, preferably cells selected from the group comprising white blood cells, saliva leukocytes, lymphoblasts, epidermal cells, and fibroblasts.


SEQUENCE LISTING (28 sequences)

SEQ ID NO: 1 - DNA, 25 bp, Artificial Sequence
  Synthetic oligonucleotide
agagatcccatcctttccatataac

SEQ ID NO: 2 - DNA, 22 bp, Artificial Sequence
  Synthetic oligonucleotide
acccgtcctatcagaaccagtg

SEQ ID NO: 3 - DNA, 23 bp, Artificial Sequence
  Synthetic oligonucleotide
acgtctcagtcctattggttacg

SEQ ID NO: 4 - DNA, 20 bp, Artificial Sequence
  Synthetic oligonucleotide
ctccctaagtttcccctcgg

SEQ ID NO: 5 - DNA, 28 bp, Artificial Sequence
  Synthetic oligonucleotide
actactgttttaaagttattgaagtgcc

SEQ ID NO: 6 - DNA, 27 bp, Artificial Sequence
  Synthetic oligonucleotide
agagctacatgttcagatatatttgcc

SEQ ID NO: 7 - DNA, 24 bp, Artificial Sequence
  Synthetic oligonucleotide
tgagtgtgcttgctgtttgatagc

SEQ ID NO: 8 - DNA, 31 bp, Artificial Sequence
  Synthetic oligonucleotide
tggttccgttaatgcatttaaatatagtttg

SEQ ID NO: 9 - DNA, 31 bp, Artificial Sequence
  Synthetic oligonucleotide
tcaatgttagtcatgaattttcaatacattg

SEQ ID NO: 10 - DNA, 26 bp, Artificial Sequence
  Synthetic oligonucleotide
acatataggcataccacaagagattc

SEQ ID NO: 11 - DNA, 24 bp, Artificial Sequence
  Synthetic oligonucleotide
tgccattgttttgctggatgtagg

SEQ ID NO: 12 - DNA, 30 bp, Artificial Sequence
  Synthetic oligonucleotide
tgatatttacccttagatgtgtattctttc

SEQ ID NO: 13 - DNA, 27 bp, Artificial Sequence
  Synthetic oligonucleotide
aggaacactgagcaagttataataagg

SEQ ID NO: 14 - DNA, 19 bp, Artificial Sequence
  Synthetic oligonucleotide
acttctgggttcccgcccc

SEQ ID NO: 15 - DNA, 25 bp, Artificial Sequence
  Synthetic oligonucleotide
aacacatctattgacattgtctccc

SEQ ID NO: 16 - DNA, 28 bp, Artificial Sequence
  Synthetic oligonucleotide
agatggtcaacatcattagttatcatgg

SEQ ID NO: 17 - DNA, 25 bp, Artificial Sequence
  Synthetic oligonucleotide
tgttgatagtctatctccactacag

SEQ ID NO: 18 - DNA, 21 bp, Artificial Sequence
  Synthetic oligonucleotide
agctgccatggaagactggac

SEQ ID NO: 19 - DNA, 26 bp, Artificial Sequence
  Synthetic oligonucleotide
aggatgcttgtgtgtaaatttacagg

SEQ ID NO: 20 - DNA, 31 bp, Artificial Sequence
  Synthetic oligonucleotide
cataaaacatgtcctaagaatttcattaaag

SEQ ID NO: 21 - DNA, 25 bp, Artificial Sequence
  Synthetic oligonucleotide
actgataggtgcttgaacaaacagg

SEQ ID NO: 22 - DNA, 24 bp, Artificial Sequence
  Synthetic oligonucleotide
agcttggctgaaactgttgcatag

SEQ ID NO: 23 - DNA, 24 bp, Artificial Sequence
  Synthetic oligonucleotide
cctttcctgtcgcttgatttgttg

SEQ ID NO: 24 - DNA, 27 bp, Artificial Sequence
  Synthetic oligonucleotide
agcagtatgtgaacaccttaaactatc

SEQ ID NO: 25 - DNA, 26 bp, Artificial Sequence
  Synthetic oligonucleotide
tgtaatctgaagtatgctagccaaag

SEQ ID NO: 26 - DNA, 23 bp, Artificial Sequence
  Synthetic oligonucleotide
tgtttttcaaggagtggagggtc

SEQ ID NO: 27 - DNA, 22 bp, Artificial Sequence
  Synthetic oligonucleotide
agggattcctccttagtcgctg

SEQ ID NO: 28 - DNA, 23 bp, Artificial Sequence
  Synthetic oligonucleotide
tgtatgctacctgctgtgacatg